(19) United States
(12) Patent Application Publication
(10) Pub. No.: US 2025/0223361 A1
(43) Pub. Date: Jul. 10, 2025

(54) HUMAN ANTIBODY OR ANTIGEN BINDING FRAGMENT THEREOF SPECIFIC AGAINST PD-L1 TO ENHANCE T-CELL FUNCTION

(71) Applicant: MABTREE BIOLOGICS AG, Basel (CH)

(72) Inventor: Ratna Monica Alexander, Basel (CH)

(73) Assignee: MABTREE BIOLOGICS AG, Basel (CH)

(21) Appl. No.: 18/853,130

(22) PCT Filed: Jul. 4, 2022
(86) PCT No.: PCT/IB2022/056172

§ 371 Date: Oct. 1, 2024

(30) Foreign Application Priority Data
Apr. 1, 2022 (IN) 202241019848

(51) Int. Cl. C07K 16/28; A61K 39/00; A61P 35/00; C07K 16/00

(52) U.S. Cl. CPC C07K 16/2827; A61P 35/00; C07K 16/005; A61K 2039/505; C07K 2317/24; C07K 2317/76; C07K 2317/92

(57) ABSTRACT

The present invention provides human antibody(ies) or antigen binding fragment(s) specific against PD-L1 capable of binding to PD-L1 and block its interaction with programmed death-1 (PD-1) thereby enhancing T-cell function which has applications in treatment of cancer and infectious diseases. The human antibodies or antigen binding fragments specific against PD-L1 comprise of LCDR1 of Seq. ID 1 or Seq. ID 2; LCDR2 of Seq. ID 3; and LCDR3 of Seq. ID 4 or Seq. ID 5; further comprise of HCDR1 of Seq. ID 8; HCDR2 of Seq. ID 9; and HCDR3 of Seq. ID 10 or Seq. ID 11. The present invention also provides human antibodies or antigen binding fragments specific against PD-L1 1 comprising light chain of Seq. ID 6 and heavy chain of Seq. ID 12, and light chain of Seq. ID 7 and heavy chain of Seq. ID 13.

### FIELD OF INVENTION

The present invention relates to antibody(ies) or antibody fragment(s) that bind to Programmed Death Ligand 1 (PD-L1), also called as cluster of differentiation 274 (CD274). Further, the invention relates to antibody or an antibody fragment that binds to PD-L1 for its application to enhance T-cell function including the treatment of cancer and acute/chronic infections.

### BACKGROUND OF THE INVENTION

Cancer is a serious health problem and one of the primary diseases leading to morbidity and mortality in the world. Cancer is unchecked cell growth leading to clonal proliferation of cancerous cells. There can be various triggers for cancer including genetic disorders, viral induced, carcinogen induced, hormone induced, environment induced, poor DNA repair mechanisms, poor function of tumor suppressor genes etc. Cancer cells incorporate numerous strategies, including those occur at the level of genetic and epigenetics that aid in their escape from immune surveillance mechanisms and allowing then to grow. There is a complex cross-talk between immune cells and cancer cells leading to either inhibition or tolerance of tumor growth. Cancer cells evade immune response via exploiting various mechanisms. One of the major mechanisms involves suppressing immune checkpoints and upregulating inhibitory immunoreceptors. Examples of inhibitory proteins are PD-L1 and PD-L2, which form complexes with PD-1 on T cells.

PD-L1 (Programmed death ligand 1, CD274/B7H1, PDCD1L1, PDCD1LG1, PDL1) is an essential immune checkpoint protein over expressed by cancer cells which binds to programmed death 1 (PD-1) on T-cells. PD-1/PD-L1 pathway controls the induction and maintenance of immune tolerance within the tumor microenvironment. PD-1 is an inhibitor of both adaptive and innate immune responses, and is expressed on activated T, natural killer (NK) and B lymphocytes, macrophages, dendritic cells (DCs) and monocytes. The binding of PD-L1 to PD-1 inhibits T cell proliferation and activity, leading to T cell immunosuppression, termed anergy or T-cell exhaustion, and as well attenuates cytotoxic secretion thereby favouring cancer to grow by mitigating anti-tumor immune responses. Therefore, inhibition of the PD-1/PD-L1 interaction between T cells and cancer, using antibodies that block this interaction, mediates potent antitumor activity of T cells in preclinical models. Some cancer cells exhibit a high amount of PD-L1, and in these cancers, anti-PD-L1 immunotherapy remains one of the most beneficial treatments in improving the therapeutic outcome of these patients.

Several antibodies targeting PD-1/PD-L1 interaction have been developed for immunotherapy against cancer/tumor.

Nivolumab is a fully human monoclonal antibody raised against PD-1 that inhibits the interaction between PD-1 and PD-L1. It has been provided as a combination therapy along with apatinib for treatment of advanced liver carcinosarcoma. However, grade 3 increase of aminotransferase levels occurred during treatment, suggesting that the combination therapy should be recommended only after risk assessment.

Likewise, pembrolizumab, is a humanized antibody against PD-1 that has been shown to be more effective in patients with lung metastases compared to those with liver metastases (62 vs. 22%, respectively). Pembrolizumab plus trastuzumab (antibody against HER2) has also been used to treat patients with PD-L1-positive, trastuzumab-resistant, and HER2-positive breast cancer.

Nonetheless, administering two antibodies can be burdensome due to different dosing and dosing interval between two antibodies, thereby causing multiple intravenous injections at different time points. Furthermore, two antibodies can have drastically different stability profiles and physical nature, e.g., variations in Fc-glycosylation, partial heavy chain C-terminal Lys processing, Fc-methionine oxidation, hinge-region cleavage, and glycation of Lys residues. Each antibody has varied physicochemical and/or thermodynamic properties, e.g., different degradation profiles when exposed to heat, freezing, light, pH extremes, agitation, sheer-stress, some metals, and organic solvents. Therefore, while a single formulation containing two antibodies would improve convenience, the unique nature of each antibody makes such a single formulation difficult to identify.

Atezolizumab is a humanized anti-PD-L1 monoclonal antibody that blocks interactions between PD-L1 and its receptors PD-1 and B7.1, thereby enhancing T-cell mediated anticancer immunity. Atezolizumab monotherapy has been approved for metastatic urothelial carcinoma and non-small cell lung cancer.

The disadvantages of targeting PD-1/PD-L1 immune checkpoint with antibody therapy is the different levels of immune-related adverse events (irAEs), which include cardiotoxicity; cytokine release syndrome; myocarditis; pneumonitis; hepatitis; thyroiditis; endocrine dysfunction; fatigue, rash, and diarrhoea; and polymyalgia rheumatic/giant cell arteritis. Another major limitation is unpredictable off-target toxicities to critical organs in children in whom the organs are less mature and potentially prone to life-long disabilities. Therefore, focus has shifted from murine or other kinds of antibodies derived from non-human mammals, to humanized antibodies. Humanized antibodies are antibodies from non-human species whose protein sequences have been modified to increase their similarity to antibody variants produced naturally in humans. Nevertheless, the use of humanized antibodies is a very high risky method for treatment of cancer. Many humanized antibodies are known to trigger the development of anti-drug antibody responses and infusion reactions. This necessitates the need to develop fully human antibodies that would minimize these safety concerns in a therapeutic context.

Further, the role of PD-1 signalling is also well studied in T lymphocytes for defending the body against pathogenic microbes as well as maintaining tolerance to self. It is well known that PD-1 signalling through T cells limits immune-mediated tissue damage during infection. As the PD-1 pathway also serves to limit the activation of self-reactive T cells, this signalling pathway plays a role in reducing the risk for autoimmunity and immunopathology. It has been discovered that T-cell dysfunction or anergy occurs concurrently with induced and sustained expression of the inhibitory receptor, PD-1. Hence, therapeutic targeting PD-1 and its signalling partner PD-L1 are an area of interest in these pathophysiological conditions.

However, an optimal therapeutic method to target this pathway is yet to be found and commercialized.

The present invention takes into consideration the drawbacks of the prior art and provides a novel fully human monoclonal antibody against or an antibody fragment thereof that binds to PD-L1 for its application in treating cancer.

### OBJECT(S) OF THE INVENTION

The main object of the present invention is to provide a novel human antibody(ies) or antibody fragment(s) against PD-L1 for use in enhancing T-cell function and having an application for the treatment of cancer and infectious diseases, acute or chronic.

Another object of the present invention is to provide a nucleic acid encoding the antibody or an antigen-binding fragment thereof against PD-L1.

Yet another object of the present invention to provide a composition for preventing or treating cancer, containing the antibody or an antigen-binding fragment thereof.

Yet another object of the present invention to provide a composition for preventing or treating infectious diseases, containing the antibody or an antigen-binding fragment thereof.

### SUMMARY OF THE INVENTION

In one aspect, the present invention provides antibodies and antigen binding fragments thereof that bind to programmed death ligand 1 (PD-L1). Said antibodies and antigen binding fragments thereof are human antibodies. Said antibodies and antibody fragments thereof bind to PD-L1 and block its interaction with programmed death-1 (PD-1). The binding of said antibodies against PD-L1 and antibody fragments thereof to PD-1 disrupts the PD-1/PD-L1 pathway thereby enhancing T-cell function. Said antibodies against PD-L1 and antibody fragments thereof have application as therapeutic agents in treatment of cancer and infectious diseases.

In one embodiment of the invention, the invention provides human antibodies against PD-L1 and antibody fragments thereof comprising of:
a) a light chain variable region comprising of LCDR1, LCDR2, and LCDR3 sequences, wherein,
LCDR1 is amino acid sequence selected from the group consisting of Seq. ID 1 or Seq. ID 2;
LCDR2 is amino acid sequence of Seq. ID 3; and
LCDR3 is amino acid sequence selected from the group consisting of Seq. ID 4 or Seq. ID 5;
and
b) a heavy chain variable region comprising of HCDR1, HCDR2, and HCDR3 sequences, wherein,
HCDR1 is amino acid sequence of Seq. ID 8;
HCDR2 is amino acid sequence of Seq. ID 9; and
HCDR3 is amino acid sequence selected from the group consisting of Seq. ID 10 or Seq. ID 11.

In another embodiment, the invention provides human antibody against PD-L1 and antibody fragment thereof, wherein, said antibody and antibody fragment thereof comprises light chain variable region consisting of

LCDR1 of Seq. ID 1,

LCDR2 is of Seq. ID 3, and

LCDR3 is of Seq. ID 4.

In yet another embodiment, the invention provides human antibody against PD-L1 and antibody fragment thereof, wherein, said antibody and antibody fragment comprises light chain variable region consisting of

LCDR1 of Seq. ID 2,

LCDR2 is of Seq. ID 3, and

LCDR3 is of Seq. ID 5.

Further, in yet another embodiment, the invention provides human antibody against PD-L1 and antibody fragment thereof, wherein, said antibody and antibody fragment comprises heavy chain variable region consisting of

HCDR1 of Seq. ID 8,

HCDR2 is of Seq. ID 9, and

HCDR3 is of Seq. ID 10.

In yet another embodiment, the invention provides human antibody against PD-L1 and antibody fragment thereof, wherein, said antibody and antibody fragment comprises heavy chain variable region consisting of

HCDR1 of Seq. ID 8,

HCDR2 is of Seq. ID 9, and

HCDR3 is of Seq. ID 11

Furthermore, in another embodiment, the invention provides human antibody against PD-L1 and antibody fragment thereof, wherein, said antibody and antibody fragment comprises light chain of amino acid sequence selected from the group consisting of Seq. ID 6 and Seq. ID 7; and

the heavy chain is amino acid sequence selected from the group consisting of Seq. ID 12 and Seq. ID 13.

TABLE 1
List of polypeptide sequences of anti-PD-L1
antibody light chains, heavy chains
and the CDRs of light and heavy chains

| | Seq. | |
|---|---|---|
| Name | ID | Amino acid sequence |
| LCDR1 | 1 | TGSSSNIGAGYDVH |
| LCDR1 | 2 | FGSSSNIGAGYDVH |
| LCDR2 | 3 | YGNNNRPS |
| LCDR3 | 4 | QSYDSSLSGSV |
| LCDR3 | 5 | GSYDSSLSGSV |
| AHP01679 | 6 | MGWSCIILFLVATATGVHSQAVLTQPS |
| LC | | SVSGAPGQRVTISCTGSSSNIGAGYDV |
| | | HWYQQLPGTAPKLLIYGNNNRPSGVPD |
| | | RFSGSKSGTSASLAITGLQAEDEADYY |
| | | CQSYDSSLSGSVFGGGTKLTVLGQPKA |
| | | APSVTLFPPSSEELQANKATLVCLISD |
| | | FYPGAVTVAWKADSSPVKAGVETTTPS |
| | | KQSNNKYAASSYLSLTPEQWKSHRSYS |
| | | CQVTHEGSTVEKTVAPTECS |
| QJ001-45 | 7 | QAVLTQPSSVSGAPGQRVTISCFGSSS |
| LC | | NIGAGYDVHWYQQLPGTAPKLLIYGNN |
| | | NRPSGVPDRFSGSKSGTSASLAITGLQ |
| | | AEDEADYYCGSYDSSLSGSVFGGGTKL |
| | | TVLGQPKAAPSVTLFPPSSEELQANKA |
| | | TLVCLISDFYPGAVTVAWKADSSPVKA |
| | | GVETTTPSKQSNNKYAASSYLSLTPEQ |
| | | WKSHRSYSCQVTHEGSTVEKTVAPTEC |
| | | S |
| HCDR1 | 8 | GGTFSSYAIS |
| HCDR2 | 9 | WMGRIIPILGIAN |
| HCDR3 | 10 | TTFIQQWLYAFDI |
| HCDR3 | 11 | TTFFQQWLYAFDI |
| AHP01679 | 12 | MGWSCIILFLVATATGVHSEVQLVQSG |
| HC | | AEVKKPGSSVKVSCKASGGTFSSYAIS |
| | | WVRQAPGQGLEWMGRIIPILGIANYAQ |
| | | KFQGRVTITADKSTSTAYMELSSLRSE |
| | | DTAVYYCTTFIQQWLYAFDIWGQGTMV |
| | | TVSSASTKGPSVFPLAPSSKSTSGGTA |
| | | ALGCLVKDYFPEPVTVSWNSGALTSGV |
| | | HTFPAVLQSSGLYSLSSVVTVPSSSLG |
| | | TQTYICNVNHKPSNTKVDKKVEPKSCD |
| | | KTHTCPPCPAPELLGGPSVFLFPPKPK |
| | | DTLMISRTPEVTCVVVDVSHEDPEVKF |
| | | NWYVDGVEVHNAKTKPREEQYNSTYRV |
| | | VSVLTVLHQDWLNGKEYKCKVSNKALP |
| | | APIEKTISKAKGQPREPQVYTLPPSRD |
| | | ELTKNQVSLTCLVKGFYPSDIA VEWE |
| | | SNGQPENNYKTTPPVLDSDGSFFLYSK |
| | | LTVDKSRWQQGNVFSCSVMHEALHNHY |
| | | TQKSLSLSPGK |
| QJ001-45 | 13 | EVQLVQSGAEVKKPGSSVKVSCKASGG |
| HC | | TFSSYAISWVRQAPGQGLEWMGRIIPI |
| | | LGIANYAQKFQGRVTITADKSTSTAYM |
| | | ELSSLRSEDTAVYYCTTFFQQWLYAFD |
| | | IWGQGTMVTVSSASTKGPSVFPLAPSS |
| | | KSTSGGTAALGCLVKDYFPEPVTVSWN |
| | | SGALTSGVHTFPAVLQSSGLYSLSSVV |
| | | TVPSSSLGTQTYICNVNHKPSNTKVDK |
| | | KVEPKSCDKTHTCPPCPAPELLGGPSV |
| | | FLFPPKPKDTLMISRTPEVTCVVVDVS |
| | | HEDPEVKFNWYVDGVEVHNAKTKPREE |
| | | QYNSTYRVVSVLTVLHQDWLNGKEYKC |
| | | KVSNKALPAPIEKTISKAKGQPREPQV |
| | | YTLPPSRDELTKNQVSLTCLVKGFYPS |
| | | DIA VEWESNGQPENNYKTTPPVLDSD |
| | | GSFFLYSKLTVDKSRWQQGNVFSCSVM |
| | | HEALHNHYTQKSLSLSPGK |

In yet another embodiment, the invention provides human antibody against PD-L1 and antibody fragment thereof, wherein, said antibody and antibody fragment comprises light chain of Seq. ID 6 and heavy chain of Seq. ID 12. Said human antibody against PD-L1 and antibody fragment thereof having EC50 value of 0.1629 μg/ml compared to EC50 value 0.1052 of commercially available anti-PD-L1 antibody, Atezolizumab.

In yet another embodiment, the invention provides human antibody against PD-L1 and antibody fragment thereof, wherein, said antibody and antibody fragment comprises light chain of Seq. ID 7 and heavy chain of Seq. ID 13. Said human antibody against PD-L1 and antibody fragment thereof having EC50 value 0.0966 μg/ml compared to EC50 value 0.1052 of commercially available anti-PD-L1 antibody, Atezolizumab.

In yet another embodiment, the invention provides human antibodies against PD-L1 and antibody fragments thereof compete with any of the exemplary antibodies provided herein for binding to PD-L1 which may be measured by ELISA, surface plasma resonance (SPR) assay, or any other method known in art.

### BRIEF DESCRIPTION OF THE DRAWINGS

The object of the invention may be understood in more details and more particularly description of the invention briefly summarized above by reference to certain embodiments thereof which are illustrated in the appended drawings, which drawings form a part of this specification. It is to be noted, however, that the appended drawings illustrate preferred embodiments of the invention and are therefore not to be considered limiting of its scope, for the invention may admit to other equally effective equivalent embodiments.

FIG. 1 illustrates FACS analysis of 88 selected clones for cell binding affinity towards target antigen (human PD-L1.Fc), and human IgG as control; PC was a positive cell binder, a positive antibody control, NC means non-related Fab-phage samples as negative control, Blank was only medium control, grey shaded and bold cells of the table the clones which showed cell binding affinity towards target antigen;

FIG. 2 shows a result of SDS-PAGE analysis to identify purity of PD-L1 antibodies of 19 selected candidates;

FIG. 3 is a table showing the ELISA EC50 values of the selected purified IgGs of PD-L1 antibodies, where Atezolizumab was used as positive control (PC), and human IgG (HuIgG) was negative control;

FIG. 4A-4H are graphical representations of PD-1/PD-L1 blockade assays exhibited by different parental anti-PD-L1 antibodies, where positive control antibody is Atezolizumab, and negative control antibody IgG1;

FIG. 5 depicts a schematic of the experimental strategy to determine the therapeutic activity of anti-PD-L1 antibodies using mouse model;

FIG. 6A is a graphical representation of effect of anti-PD-L1 antibody AHP01679 (parental) compared to positive control antibody Atezolizumab, and negative control antibody IgG1 in tumor growth in mouse model;

FIG. 6B is a graphical representation of effect of affinity matured anti-PD-L1 antibodies QJ001-13 and QJ001-45 derived from parental antibody AHP01679, compared to positive control antibody Atezolizumab, and negative control antibody IgG1 in tumor growth in mouse model; and

FIG. 7 is graphical representations of PD-1/PD-L1 blockade assays comparing the functionality of parental anti-PD-L1 antibody AHP01679, affinity matured anti-PD-L1 antibodies QJ001-45, positive control antibody Atezolizumab, and negative control antibody IgG1.

### DETAILED DESCRIPTION OF THE INVENTION

The present invention now will be described hereinafter with reference to the detailed description, in which some, but not all embodiments of the invention are indicated. Indeed, the invention may be embodied in many different forms and should not be construed as limited to the embodiments set forth herein; rather, these embodiments are provided so that this disclosure will satisfy applicable legal requirements. The present invention is described fully herein with non-limiting embodiments and exemplary experimentation.

### Definitions

The term “antibody” as used herein includes any immunoglobulin, monoclonal antibody, polyclonal antibody that binds to a specific antigen. A native intact antibody comprises two heavy chains (H) and two light chains (L). Each heavy chain consists of a variable region (V_{H}) and a first, second, and third constant region (C_{H}), while each light chain consists of a variable region (V_{L}) and a constant region (C_{L}). The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The constant regions of the heavy and light chains are not involved in antigen binding, but exhibit various effector functions.

The term “CDR or complementarity determining regions” as used herein includes parts of variable region in both chains of antibody. These generally are three highly variable loops in each of the light and heavy chains. Light (L) chain CDRs include LCDR1, LCDR2, and LCDR3, and heavy (H) chain CDRs include HCDR1, HCDR2, HCDR3).

The term “antigen-binding fragment” as used herein refers to an antibody fragment formed from a portion of an antibody comprising one or more CDRs, or any other antibody fragment that binds to an antigen but does not comprise an intact native antibody structure. An antigen-binding fragment is capable of binding to the same antigen to which the parent antibody binds.

The term “fully human antibody” or “human antibody” as used herein, with reference to antibody or antigen-binding fragment, means that the antibody or the antigen-binding fragment has or consists of amino acid sequence(s) corresponding to that of an antibody produced by a human or a human immune cell, or derived from a non-human source such as a transgenic non-human animal that utilizes human antibody repertoires or other human antibody-encoding sequences. In certain embodiments, a fully human antibody does not comprise amino acid residues (in particular antigen-binding residues) derived from a non-human antibody.

The term “humanized antibody” as used herein, with reference to antibody or antigen-binding fragment, means that the antibody is derived from non-human species whose amino acid sequences have been modified to increase their similarity to antibody variants produced naturally in humans.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen.

The term “PD-L1” as used herein refers to programmed cell death ligand 1 also known as CD274/B7H1, PDCD1L1, PDCD1LG1, PDL1. PD-L1 is a 40 kDa transmembrane protein expressed on various tumor cells, stromal cells or both, and binds to PD-1.

The term “PD-1”, as used herein refers to programmed cell death-1 also called as CD279, is known as a key immune-checkpoint receptor expressed by activated T cells, which mediates immunosuppression. Inhibition of the interaction between PD-1 and PD-L1 can enhance T-cell responses and thus mediates anti-cancer activity.

The term “Anti-PD-L1 antibody” as used herein refers to an antibody that is capable of specific binding to PD-L1 (e.g. human PD-L1) with an affinity which is sufficient to provide for diagnostic and/or therapeutic use.

The ability to “block binding” as used herein refers to the ability of an antibody or antigen-binding fragment to inhibit the binding interaction between two molecules (e.g. human PD-L1 and an anti-PD-L1 antibody) to any detectable degree.

The term “EC50” as used herein refers to the concentration of antibody required to a block binding of PD-1/PD-L1 by 50% of the maximum binding.

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating.

In one aspect, the present invention provides anti-PD-L1 human antibodies and the antigen-binding fragments thereof. Said antibodies inhibit or block the interaction between PD-1 and PD-L1 to enhance T-cell responses and thus mediate anti-cancer activity.

In one of the embodiments, the present invention provides human anti-PDL-1 antibody, AHP01679 or the parental antibody, comprising

a) light chain of Seq. ID 6 comprising of LCDR1 of Seq. ID 1, LCDR2 of Seq. ID 3, and LCDR3 of Seq. ID 4,

and

b) heavy chain of Seq. ID 12 comprising of HCDR1 of Seq. ID 8, HCDR2 of Seq. ID 9, and HCDR3 of Seq. ID 10.

In another embodiments, the present invention provides affinity matured human anti-PDL-1 antibody, QJ001-45, derived from AHP01679 antibody, comprising

a) light chain of Seq. ID 7 comprising of LCDR1 of Seq. ID 2, LCDR2 of Seq. ID 3, and LCDR3 of Seq. ID 5,

and

b) heavy chain of Seq. ID 13 comprising of HCDR1 of Seq. ID 8, HCDR2 of Seq. ID 9, and HCDR3 of Seq. ID 11.

The sequence IDs of Anti-PD-L1 antibodies and their CDRs as summarized in Table 2.

TABLE 2
Anti-PD-L1 antibodies and their light chain and heavy chain CDRs

| Anti-PD-L1 | | CDR1 | CDR2 | CDR3 |
|---|---|---|---|---|
| AHP01679 | Light Chain | LCDR1 of | LCDR2 of | LCDR3 of |
| (Parental) | of Seq. ID 6 | Seq. ID 1 | Seq. ID 3 | Seq. ID 4 |
| | Heavy Chain | HCDR1 of | HCDR2 of | HCDR3 of |
| | of Seq. ID 12 | Seq. ID 8 | Seq. ID 9 | Seq. ID 10 |
| QJ001-45 | Light Chain | LCDR1 of | LCDR2 of | LCDR3 of |
| (Affinity | of Seq. ID 7 | Seq. ID 2 | Seq. ID 3 | Seq. ID 5 |
| matured from | Heavy Chain | HCDR1 of | HCDR2 of | HCDR3 of |
| parental | of Seq. ID 13 | Seq. ID 8 | Seq. ID 9 | Seq. ID 11 |
| antibody) | | | | |

In another embodiment, the present invention provides the nucleotide sequences encoding light chains and heavy chains of AHP01679 (Parental) antibody, wherein,

Seq. ID 14 encodes the light chain of Seq. ID 6, and

Seq. ID 15 encodes the heavy chain of Seq. ID 12.

In yet another embodiment, the present invention provides the nucleotide sequences encoding light chains and heavy chains of QJ001-45 (Affinity matured from parental antibody) antibody, wherein,

Seq. ID 16 encodes the light chain of Seq. ID 7, and

Seq. ID 17 encodes the heavy chain of Seq. ID 13.

TABLE 3
Nucleotide sequences encoding light and
heavy chains of anti-PD-L1 antibodies

| | Nucleotide sequence |
|---|---|
| Name | and Seq. ID no. |
| AHP01679 | Seq. ID 14 |
| (Parental) | CAGGCCGTGCTGACCCAGCCTAGCTCC |
| Light chain | GTGTCTGGAGCACCAGGACAGAGGGTG |
| Seq. ID 6 | ACCATCAGCTGCACAGGCTCTAGCTCC |
| | AATATCGGCGCCGGCTACGACGTGCAC |
| | TGGTATCAGCAGCTGCCCGGCACAGCC |
| | CCTAAGCTGCTGATCTACGGCAACAAT |
| | AACCGGCCATCCGGCGTGCCCGATAGA |
| | TTCTCCGGCTCTAAGAGCGGCACCTCC |
| | GCCTCTCTGGCCATCACAGGACTGCAG |
| | GCAGAGGACGAGGCAGATTACTATTGT |
| | CAGTCTTATGACTCTAGCCTGAGCGGA |
| | TCCGTGTTTGGAGGAGGAACCAAGCTG |
| | ACAGTGCTGGGGCAGCCCAAGGCCGCT |
| | CCTAGCGTGACACTGTTTCCCCCTTCC |
| | TCCGAGGAGCTGCAGGCCAACAAGGCC |
| | ACCCTGGTGTGCCTGATCTCCGACTTC |
| | TATCCTGGCGCCGTGACAGTGGCCTGG |
| | AAGGCTGATTCTAGCCCAGTGAAGGCT |
| | GGCGTGGAGACCACAACCCCCTCCAAG |
| | CAGTCTAACAATAAGTATGCCGCTTCC |
| | TCTTACCTGAGCCTGACACCAGAGCAG |
| | TGGAAGTCCCACCGGTCTTACAGCTGC |
| | CAGGTCACTCACGAAGGCTCTACCGTG |
| | GAAAAGACAGTCGCACCCACCGAATGC |
| | TCATGA |
| AHP01679 | Seq. ID 15 |
| (Parental) | GAGGTGCAGCTGGTGCAGAGCGGAGCA |
| Heavy chain | GAGGTGAAGAAGCCAGGAAGCTCCGTG |
| Seq. ID 12 | AAGGTGTCCTGCAAGGCCTCTGGCGGC |
| | ACCTTCTCTAGCTACGCCATCTCCTGG |
| | GTGCGGCAGGCACCAGGACAGGGACTG |
| | GAGTGGATGGGCAGAATCATCCCTATC |
| | CTGGGCATCGCCAACTACGCCCAGAAG |
| | TTTCAGGGCAGGGTGACCATCACAGCC |
| | GACAAGTCTACCAGCACAGCCTATATG |
| | GAGCTGTCCTCTCTGCGGTCTGAGGAT |
| | ACAGCCGTGTACTATTGTACCACATTC |
| | ATCCAGCAGTGGCTGTATGCCTTTGAC |
| | ATCTGGGGCCAGGGCACCATGGTGACA |
| | GTGAGCTCCGCCAGCACCAAGGGCCCT |
| | TCCGTGTTTCCACTGGCCCCCTCCTCT |
| | AAATCCACATCTGGCGGCACCGCCGCC |
| | CTGGGCTGTCTGGTGAAGGACTACTTC |
| | CCAGAGCCTGTGACAGTGTCCTGGAAC |
| | TCTGGCGCCCTGACATCCGGCGTGCAC |
| | ACATTTCCAGCCGTGCTGCAGAGCTCC |
| | GGCCTGTACAGCCTGTCTAGCGTGGTG |
| | ACAGTGCCCTCCTCTAGCCTGGGCACA |
| | CAGACCTATATCTGCAACGTGAATCAC |
| | AAGCCAAGCAATACCAAGGTGGACAAG |
| | AAGGTGGAGCCCAAGTCCTGTGATAAG |
| | ACACACACCTGCCCCCCTTGTCCTGCT |
| | CCCGAGCTGCTGGGCGGCCCTAGCGTG |
| | TTCCTGTTTCCACCCAAGCCTAAGGAC |
| | ACCCTGATGATCTCCCGGACACCCGAG |
| | GTGACCTGCGTGGTGGTGGACGTGTCT |
| | CACGAGGATCCTGAGGTGAAGTTCAAC |
| | TGGTATGTGGATGGCGTGGAGGTGCAC |
| | AATGCCAAGACCAAGCCCAGAGAGGAG |
| | CAGTACAACTCTACATATAGGGTGGTG |
| | AGCGTGCTGACCGTGCTGCACCAGGAC |
| | TGGCTGAACGGCAAGGAGTATAAGTGC |
| | AAGGTGTCCAATAAGGCCCTGCCCGCC |
| | CCCATCGAGAAGACAATCAGCAAGGCC |
| | AAGGGCCAGCCTCGGGAGCCACAGGTG |
| | TACACCCTGCCTCCATCCAGAGACGAG |
| | CTGACAAAGAACCAGGTGTCTCTGACA |
| | TGTCTGGTGAAGGGCTTCTATCCTAGC |
| | GATATCGCCGTGGAGTGGGAGTCCAAT |
| | GGCCAGCCAGAGAACAATTACAAGACC |
| | ACACCCCCTGTGCTGGACTCCGATGGC |
| | TCCTTCTTTCTGTATTCCAAGCTGACC |
| | GTGGATAAGTCTCGGTGGCAGCAGGGC |
| | AACGTGTTCAGCTGTTCCGTGATGCAC |
| | GAAGCCCTGCATAATCACTATACTCAG |
| | AAATCCCTGTCCCTGTCACCTGGAAAG |
| | TGA |
| QJ001-45 | Seq. ID 16 |
| (Affinity | CAGGCTGTTCTGACCCAGCCTTCCTCT |
| matured | GTGTCTGGTGCTCCTGGCCAGAGAGTG |
| from | ACCATCTCTTGTTTTGGCTCCTCCTCC |
| parental | AATATCGGCGCTGGCTACGACGTGCAC |
| antibody) | TGGTATCAGCAGCTGCCTGGCACAGCT |
| Light chain | CCCAAACTGCTGATCTACGGCAACAAC |
| Seq. ID 7 | AACCGGCCTTCTGGCGTGCCCGATAGA |
| | TTCTCCGGCTCTAAGTCTGGCACCTCT |
| | GCCAGCCTGGCTATCACTGGACTGCAG |
| | GCTGAGGACGAGGCCGACTACTACTGC |
| | GGGTCCTACGACTCTTCCCTGTCCGGC |
| | TCTGTTTTTGGCGGAGGCACCAAGCTG |
| | ACAGTGCTGGGACAGCCTAAGGCTGCC |
| | CCTTCCGTGACACTGTTCCCTCCATCC |
| | TCTGAGGAACTGCAGGCCAACAAGGCT |
| | ACCCTCGTGTGCCTGATCTCCGACTTT |
| | TACCCTGGCGCTGTGACCGTGGCCTGG |
| | AAGGCTGATAGTTCTCCTGTGAAGGCC |
| | GGCGTGGAAACCACCACACCTTCCAAG |
| | CAGTCCAACAACAAATACGCCGCCTCC |
| | TCCTACCTGTCTCTGACCCCTGAACAG |
| | TGGAAGTCCCACCGGTCCTACAGCTGC |
| | CAAGTGACCCATGAGGGCTCCACCGTG |
| | GAAAAGACCGTGGCTCCTACCGAGTGC |
| | TCT |
| QJ001-45 | Seq. ID 17 |
| (Affinity | GAAGTTCAGCTGGTTCAGTCTGGCGCC |
| matured | GAAGTGAAGAAACCTGGCTCCTCCGTG |
| from | AAGGTGTCCTGCAAGGCTTCTGGCGGC |
| parental | ACCTTCTCCTCTTACGCCATCTCCTGG |
| antibody) | GTCCGACAGGCTCCTGGACAAGGCTTG |
| Heavy chain | GAGTGGATGGGCAGAATCATCCCCATC |
| Seq. ID 13 | CTGGGAATCGCCAACTACGCCCAGAAA |
| | TTCCAGGGCAGAGTGACCATCACCGCC |
| | GACAAGTCTACCTCCACCGCCTACATG |
| | GAACTGTCCAGCCTGAGATCTGAGGAC |
| | ACCGCCGTGTACTACTGCACCACCTTC |
| | TTTCAGCAGTGGCTGTACGCCTTCGAC |
| | ATCTGGGGCCAGGGCACAATGGTCACC |
| | GTGTCCTCTGCTTCCACCAAGGGCCCC |
| | TCCGTGTTCCCCCTGGCTCCCTCTTCC |
| | AAGAGCACCAGCGGCGGCACCGCTGCT |
| | CTGGGATGTCTGGTGAAGGACTACTTC |
| | CCTGAGCCTGTGACCGTGTCCTGGAAT |
| | TCCGGCGCCCTGACCTCCGGCGTGCAC |
| | ACATTCCCTGCTGTGCTGCAGTCCTCC |
| | GGCCTGTATAGCCTGTCCTCCGTGGTG |
| | ACAGTGCCTAGCTCCAGCCTGGGCACC |
| | CAGACCTATATCTGCAACGTGAACCAC |
| | AAGCCTAGCAATACCAAGGTGGACAAG |
| | AAGGTGGAGCCTAAGAGCTGCGACAAG |
| | ACCCACACCTGTCCTCCATGTCCTGCT |
| | CCAGAACTGCTCGGCGGACCTTCCGTG |
| | TTCCTGTTTCCTCCAAAGCCTAAGGAC |
| | ACCCTGATGATCAGCAGAACCCCTGAA |
| | GTGACCTGCGTGGTGGTGGATGTGTCC |
| | CACGAGGATCCCGAAGTGAAGTTCAAT |
| | TGGTACGTGGACGGCGTGGAAGTGCAC |
| | AACGCCAAGACCAAGCCTAGAGAGGAA |
| | CAGTACAACAGCACCTACAGAGTGGTG |
| | TCCGTGCTGACCGTGCTGCACCAGGAT |
| | TGGCTGAACGGCAAAGAGTACAAGTGC |
| | AAGGTGTCCAACAAGGCCCTGCCTGCT |
| | CCTATCGAGAAAACCATCAGCAAGGCC |
| | AAGGGCCAGCCTAGGGAACCCCAGGTT |
| | TACACACTGCCTCCAAGCAGGGACGAG |
| | CTGACCAAGAATCAGGTGTCCCTGACC |
| | TGCCTGGTCAAGGGCTTCTACCCTTCC |
| | GATATCGCCGTGGAATGGGAGAGCAAT |
| | GGCCAGCCTGAGAACAACTACAAGACA |
| | ACCCCTCCTGTGCTGGACAGCGACGGC |
| | TCATTCTTCCTGTACAGCAAGCTGACA |
| | GTGGACAAGGAAGTTCAGCTGGTTCAG |
| | TCTGGCGCCGAAGTGAAGAAACCTGGC |
| | TCCTCCGTGAAGGTGTCCTGCAAGGCT |
| | TCTGGCGGCACCTTCTCCTCTTACGCC |
| | ATCTCCTGGGTCCGACAGGCTCCTGGA |
| | CAAGGCTTGGAGTGGATGGGCAGAATC |
| | ATCCCCATCCTGGGAATCGCCAACTAC |
| | GCCCAGAAATTCCAGGGCAGAGTGACC |
| | ATCACCGCCGACAAGTCTACCTCCACC |
| | GCCTACATGGAACTGTCCAGCCTGAGA |
| | TCTGAGGACACCGCCGTGTACTACTGC |
| | ACCACCTTCTTTCAGCAGTGGCTGTAC |
| | GCCTTCGACATCTGGGGCCAGGGCACA |
| | ATGGTCACCGTGTCCTCTGCTTCCACC |
| | AAGGGCCCCTCCGTGTTCCCCCTGGCT |
| | CCCTCTTCCAAGAGCACCAGCGGCGGC |
| | ACCGCTGCTCTGGGATGTCTGGTGAAG |
| | GACTACTTCCCTGAGCCTGTGACCGTG |
| | TCCTGGAATTCCGGCGCCCTGACCTCC |
| | GGCGTGCACACATTCCCTGCTGTGCTG |
| | CAGTCCTCCGGCCTGTATAGCCTGTCC |
| | TCCGTGGTGACAGTGCCTAGCTCCAGC |
| | CTGGGCACCCAGACCTATATCTGCAAC |
| | GTGAACCACAAGCCTAGCAATACCAAG |
| | GTGGACAAGAAGGTGGAGCCTAAGAGC |
| | TGCGACAAGACCCACACCTGTCCTCCA |
| | TGTCCTGCTCCAGAACTGCTCGGCGGA |
| | CCTTCCGTGTTCCTGTTTCCTCCAAAG |
| | CCTAAGGACACCCTGATGATCAGCAGA |
| | ACCCCTGAAGTGACCTGCGTGGTGGTG |
| | GATGTGTCCCACGAGGATCCCGAAGTG |
| | AAGTTCAATTGGTACGTGGACGGCGTG |
| | GAAGTGCACAACGCCAAGACCAAGCCT |
| | AGAGAGGAACAGTACAACAGCACCTAC |
| | AGAGTGGTGTCCGTGCTGACCGTGCTG |
| | CACCAGGATTGGCTGAACGGCAAAGAG |
| | TACAAGTGCAAGGTGTCCAACAAGGCC |
| | CTGCCTGCTCCTATCGAGAAAACCATC |
| | AGCAAGGCCAAGGGCCAGCCTAGGGAA |
| | CCCCAGGTTTACACACTGCCTCCAAGC |
| | AGGGACGAGCTGACCAAGAATCAGGTG |
| | TCCCTGACCTGCCTGGTCAAGGGCTTC |
| | TACCCTTCCGATATCGCCGTGGAATGG |
| | GAGAGCAATGGCCAGCCTGAGAACAAC |
| | TACAAGACAACCCCTCCTGTGCTGGAC |
| | AGCGACGGCTCATTCTTCCTGTACAGC |
| | AAGCTGACAGTGGACAAGAGCAGATGG |
| | CAGCAGGGCAACGTGTTCAGCTGCAGC |
| | GTGATGCACGAGGCCCTGCACAACCAC |
| | TACACCCAGAAGTCCCTGAGCCTGTCT |
| | CCTGGCAAA |

In another embodiment, the present invention provides constant regions of light chains of anti-PD-L1 antibodies and their antibody fragments derived from constant regions of human IgG lambda or kappa antibody. Further, the constant regions of heavy chains of anti-PD-L1 antibodies and their antibody fragments derived from constant regions of human IgG1 antibody.

In yet another embodiment, the present invention provides a method for production of recombinant monoclonal human anti-PD-L1 antibodies. The antibody may be produced by techniques known in the art. The nucleotide sequences encoding the light chains and heavy chains of antibodies are cloned in suitable vectors for expression in host cells. The method comprises the steps of:
a. Transforming suitable host cells with expression vectors containing nucleotide sequences encoding light and heavy chains of antibodies;
b. Isolating/separating/collecting antibodies produced by the host cells; and
c. Purification of antibodies.

In yet another embodiment, the present invention provides kits comprising of human anti-PD-L1 antibodies or their antibody fragments thereof useful for detection of presence of PD-L1 in a biological sample. The antibody may further be conjugated with labelling molecules. Further, the antibody may be attached to a substrate or a device for use in assays such as ELISA etc.

In yet another embodiment, the present invention provides a pharmaceutical composition comprising of human anti-PD-L1 antibodies or their antibody fragments thereof and one or more pharmaceutically acceptable carriers. The pharmaceutical compositions can be liquid solutions, suspensions, emulsions, capsules, tablets, sustained release formulation, and powder. Further, these pharmaceutical compositions may for administration as parenteral or non-parenteral routes.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### Example 1

### Screening of PD-L1 Human Antibodies Using the Human Naïve Phage Library

1. Phage display library bio-panning selection

Biotinylated Human PD-L1 Protein was used as antigen, purchased from Acro biosystems, USA. Human PD-L1.Fc Protein, and human PD-L1.his Protein were produced in-house (Genscript, USA). These were used as protein antigens to screen for PD-L1 human antibodies using human naïve phage library.

A human naïve phage display library (kappa and lambda, size: 4×1010) stock was precipitated with PEG/NaCl and re-suspended in PBS for panning. Bio-panning was carried out using soluble phase panning with biotinylated human PD-L1 protein or cell panning at round 3 .Briefly, human naïve library phage particles (input 2×1012 pfu/pool) and Dynabeads™ M280 streptavidin or HBC neutravidin strips were diluted/blocked in 3-5% BSA PBS (Phosphate Buffered saline) incubated for 1 hour with slow rotating or shaking. Then added 60-100 nM biotinylated human PD-L1 protein into the blocked libraries and incubated for 1 hour with slowly rotating or shaking. The antigen and antibody binding libraries were transferred to Dynabeads or the HBC neutravidin strip wells and incubated for 20-30 min with slowly rotating or shaking. After capture, unbound/non-specifically bound phages were removed by decanting and washing with 0.05% PBST for 8-12 times and 3-5 times with PBS.

For cell panning, CHO-human PD-L1 cell line was used as target cell, while CHO-K1 cell used as negative cell to adsorb the non-specific binders. Briefly, approximately 1×10⁷ CHO-human PD-L1 cells for each pool were collected, washed in PBS then blocked in 1 mL 3% M PBS, rotated for 1 hour at room temperature. Concurrently, 1×10¹² phage particles of each pool from the rescued library stock were also blocked in 3% M PBS, added 1×10⁷ washed CHO-K1 cells into each pool and incubated with rotation for 45 min at room temperature. The phage/cell suspension were centrifuged at 500 g for 3 min, the depleted phage were then transferred to the CHO-human PD-L1 cells and incubated for 1 hour at room temperature with rotation. The phage/cell suspension were centrifuged at 500 g for 3 min, then washed 8 times using 1% BSA-PBS wash buffer. The bound phage (on the beads or wells or cells) were eluted by triethylamine (TEA) and neutralized with 1M Tris-HCl (pH7.4). The phage eluate was used to infect 10 ml exponentially growing E. coli TG1 at 37° C. Phage particles were prepared for subsequent rounds of bio-panning, by amplification and rescued using M13K07helper phage as per standard procedures. The former round amplified phage was used as the input phage for next round bio-panning. The infected E. coli TG1 were plated on the LB-Amp+plates at round 2 and 3. Single colonies were picked and binding activity were validated by monoclonal phage ELISA and FACS.

Group 1: Soluble phase panning with biotinylated-Human PD-L1 at round 1-3.

Group 2: Soluble phase panning with biotinylated-Human PD-L1 at round 1-2. For obtaining cell binders, cell panning with CHO-PD-L1 and depletion with CHO-K1 at round 3 were carried out.

TABLE 4
Panning streamline and results summary

| | | | Input | Output | Ag Positive |
|---|---|---|---|---|---|
| Pool | Strategy | Antigen | (pfu) | (pfu) | Rate* |
| Library | Soluble | Biotinylated- | 2.0 × 10¹² | 1.2 × 10⁷ | NA |
| phage | phase | Human PD-L1 | 2.0 × 10¹² | 7.0 × 10⁶ | NA |
| | | (100 nM/beads) | | | |
| 1^{st} | Soluble | Biotinylated- | 2.0 × 10¹² | 1.7 × 10⁷ | 10/94 = 10.6% |
| amplified | phase | Human PD-L1 | 2.0 × 10¹² | 1.6 × 10⁷ | 26/94 = 27.7% |
| phage | | (100 nM/8 wells) | | | |
| 2^{nd} | Soluble | Biotinylated | 2.0 × 10¹² | 4.0 × 10⁸ | 88/94 = 94.5% |
| amplified | phase | Human PD-L1 | 2.0 × 10¹² | 4.0 × 10⁸ | 87/95 = 92.6% |
| phage | | (100 nM/beads) | | | |
| 2^{nd} | Cell | CHO-human PD- | 2.0 × 10¹² | 2.0 × 10⁹ | 89/94 = 94.7% |
| amplified | panning | L1 cell line | 2.0 × 10¹² | 2.0 × 10⁹ | 88/94 = 94.5% |
| phage | | (1 × 10⁷ cells/pool) | | | |

Positive clone*, OD450 nm value (target antigen binder) >0.5, while control value <0.3.

### 2. Monoclonal Phage ELISA Screening and DNA Sequencing

Individual colonies were grown in 96-deep-well plates and rescued by M13KO7 helper phage at 30° C. overnight. More than 90 colonies were picked of each pool (kappa and lambda) and validated by monoclonal phage ELISA.

Meantime, two 96-well ELISA microtiter plates for each pool were coated with 1 μg/ml target antigen (human PD-L1.Fc) and human IgG (as control) in coating buffer overnight at 4° C. The plates were blocked with 3-5% M PBS. After rinsing, 50 μl Fab-phage supernatant from each overnight cultured deep wells and 50 μl 0.1% PBST were added to the plates for 2 hours incubation at room temperature. After rinsing plates three times with wash buffer, the HRP-conjugated anti-M13 monoclonal antibody was added to the plates for 45 minutes at room temperature. The plates were washed for additional 6 times and the substrate solution were added to the wells for developing reaction. The absorption was measured at 450 nm using a spectrometer. Consideration of the antigen binders were much more, DNA sequencing were performed. Then all unique binders and all antigen binders of round 2 were FACS tested for confirmation. Of the 90 clones selected, 88 were chosen for further studies. The 88 clones were named as AHP series ranging.

### 3. Phage FACS Screening

The phage supernatants of antigen protein binders were tested for their binding activity to CHO-human PD-L1 cell and CHO-K1 cell (as control) using flow cytometry. Cells were washed in PBS, aliquot 100 μl/well 1×10⁵ cells/reaction and added 50 μl/well supernatant sample for incubation for 40 min at 4° C. Washed cell twice with cold PBS, then added 100 μl/well Anti-Fc Bacteriophage-Biotin (3 μg/ml) and incubated for 40 min at 4° C. Added 100 μl/well SA-iFluor 647 (1 μg/ml) and incubated for 30 min at 4° C. after twice wash. After twice washed, cells were loaded and then the fluorescence was detected. The data were collected by Flow cytometry.

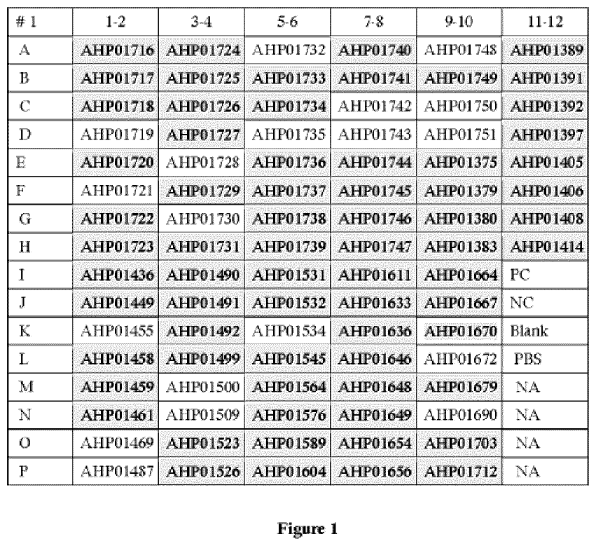

As depicted in FIG. 1, of 88 clones selected 69 clones (grey shaded and bold) showed cell binding activity as determined by FACS analysis.

Of these 19 best candidates were chosen for further analysis.

### Example 2

### Construction and production of IgGs

DNA of the 19 best cell binding candidates were synthesized and inserted into pcDNA3.4 to make expression plasmids of full-length IgGs, fully human IgGs. The heavy and light chain expression plasmids were used to co-transfect Expi293F cells. The recombinant IgGs secreted to the medium were purified using protein A affinity chromatography. The concentration and purified protein were determined by OD280 and SDS-PAGE, respectively. Binding confirmation were tested by ELISA EC50 and FACS EC50.

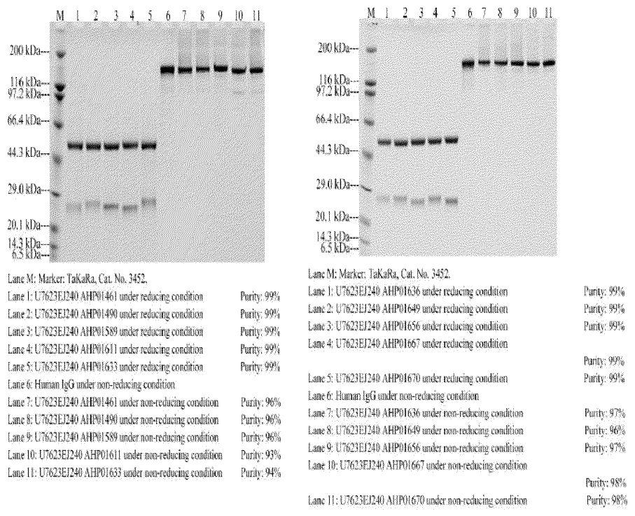

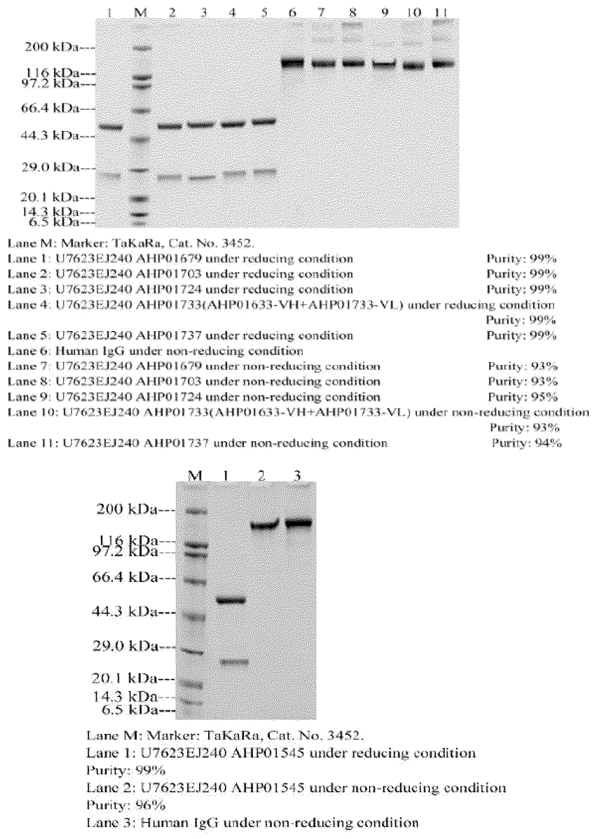

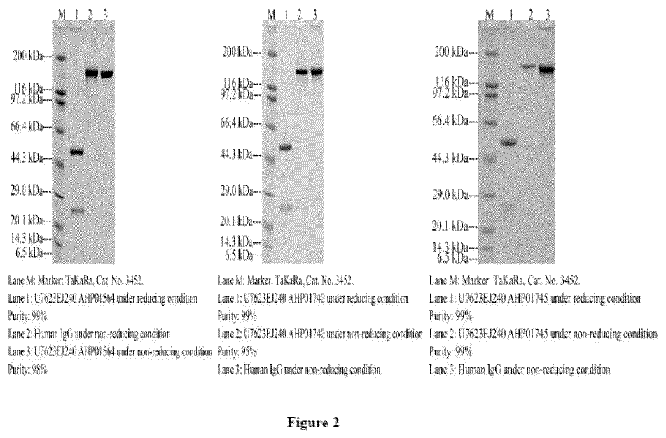

As depicted in FIG. 2, the purified IgGs migrated as ˜150 kDa band in SDS-PAGE under non-reducing conditions, and ˜50 kDa and ˜25 kDa bands under reducing conditions where the heavy and light chains were separated. The purity of fully human IgGs were all over 93%.

### Example 3

### Analysis of human IgGs

### 1. ELISA EC50 Determination of Fully Human IgGs

To evaluate the fully human IgGs binding activity to human PD-L1 protein, ELISA EC50 determination was performed. Briefly, human PD-L1.his protein was coated with 0.5 μg/ml in CBS, 100 μl/well at 4° C., incubated for overnight. Washed three times and blocking with 3% BSA-PBS at 37° C. for 1 hour. Then diluted fully human IgGs in 1% BSA-PBS were added to the blocked wells. Atezolizumab Biosimilar used as positive control, while non-related full human IgG as negative control. Incubation at room temperature for 2 hours. After rinsing plates three times with wash buffer, the Goat Anti-Human IgG (H+L)-HRP (Cat: 109-035-088, Jackson) monoclonal antibody was added to the plates for 45 minutes at room temperature. The plates were washed for additional 6 times and the substrate solution were added to the wells for developing reaction. The absorption was measured at 450nm using a spectrometer.

FIG. 3 shows the ELISA EC50 values of the selected purified IgGs. Atezolizumab was used as positive control (PC), and human IgG (HuIgG) was negative control.

### 2. FACS EC50 Determination of Fully Human IgGs

To evaluate the fully human IgGs binding activity affinity to CHO-human PD-L1 cell, FACS EC50 determination were performed, while CHO-KI as negative control cell. Fully human IgGs were serial diluted to determinate, while non-related fully human IgG as isotype control. Alexa Fluor® 647 AffiniPure Goat Anti-Human IgG (H+L) (Jackson, 109605-088, 1 ug/ml) as second antibody. Cells were loaded and then the fluorescence was detected. The data were collected by Flow cytometry.

### 3. Affinity Measurement of Purified Human IgGs

The affinity of purified antibody binding to antigen was individually determined using a Surface Plasmon Resonance (SPR) biosensor, BiacoreT200 (GE Healthcare). Antibodies were immobilized on the sensor chip through Fc capture method. Antigen was used as the analyte. The data of dissociation (kd) and association (ka) rate constants were obtained using Biacore T200 evaluation software. The equilibrium dissociation constants (KD) were calculated from the ratio of kd over ka.

### Table 5 Shows the Affinity of Purified Antibodies Towards Antigen

| | | | | | | Rmax |
|---|---|---|---|---|---|---|
| Ligand | Analyte | Chi² (RU²) | ka (1/Ms) | kd(1/s) | KD (M) | (RU) |
| AHP01636 | PD-L1 | 5.43E−01 | 1.05E+06 | 5.09E−04 | 4.85E−10 | 52 |
| AHP01740 | PD-L1 | 7.86E−01 | 1.19E+06 | 2.74E−03 | 2.30E−09 | 74.2 |
| AHP01679 | PD-L1 | 6.88E−01 | 6.35E+05 | 2.59E−03 | 4.09E−09 | 71.8 |
| AHP01745 | PD-L1 | 3.41E−02 | 9.46E+04 | 4.45E−04 | 4.70E−09 | 15.9 |
| AHP01667 | PD-L1 | 4.53E−01 | 4.02E+05 | 2.12E−03 | 5.27E−09 | 78.2 |
| AHP01670 | PD-L1 | 2.54E−01 | 1.11E+05 | 8.92E−04 | 8.03E−09 | 63.8 |
| AHP01589 | PD-L1 | 5.86E−02 | 1.21E+05 | 1.03E−03 | 8.52E−09 | 12.9 |
| AHP01490 | PD-L1 | 2.98E−01 | 9.51E+05 | 9.58E−03 | 1.01E−08 | 57.8 |
| AHP01656 | PD-L1 | 3.82E−01 | 1.01E+06 | 1.33E−02 | 1.31E−08 | 58.9 |
| AHP01545 | PD-L1 | 1.16E+00 | 5.75E+05 | 9.44E−03 | 1.64E−08 | 66.9 |
| AHP01649 | PD-L1 | 9.81E−02 | 3.34E+04 | 1.34E−03 | 4.00E−08 | 106 |
| AHP01737 | PD-L1 | 4.29E−01 | 5.42E+05 | 1.40E−01 | 2.59E−07 | 171.4 |
| AHP01461 | PD-L1 | 5.41E+00 | 2.86E+06 | 8.75E−01 | 3.06E−07 | 72.9 |
| AHP01703 | PD-L1 | 2.07E−01 | 1.40E+05 | 4.31E−02 | 3.07E−07 | 83.8 |
| AHP01724 | PD-L1 | 3.85E+00 | 4.31E+05 | 1.41E−01 | 3.27E−07 | 135.4 |
| AHP01633 | PD-L1 | 1.43E+00 | 4.20E+05 | 1.56E−01 | 3.72E−07 | 130.1 |

Of selected 19 candidates of fully human IgGs only 16 showed affinity to human PD-L1.his in the range of 0.1-100 nanomolar level. Three IgG candidates did not show affinity.

Nine fully human IgGs against PD-L1 (AHP01636, AHP01740, AHP01679, AHP01667, AHP01670, AHP01490, AHP01656, AHP01545 and AHP01589) with an affinity between 10-0.1 nM were studied for functional validation as detailed below in the PD1/PD-L1 blockade assay.

### 4. PD1/PD-L1 Blockade Bioassay

The nine fully human IgGs against PD-L1 (AHP01636, AHP01740, AHP01679, AHP01667, AHP01670, AHP01490, AHP01656, AHP01545 and AHP01589) were tested for functional activity by PD1/PD-L1 blockade bioassay.

GS-J2/PD-1 were effector cells, which were Jurkat T cells expressing human PD-1 and NFAT-mediated luciferase, used for the assay. GS-C3/PD-L1 were target cells, which were CHO-K1 cells expressing human PD-L1 and a cell surface protein capable of activating T-cell receptors (TCR). Atezolizumab biosimilar was used as the positive control and the blockade activities of selected fully human IgGs against PD-L1 were evaluated by PD1/PD-L1 Blockade Assay.

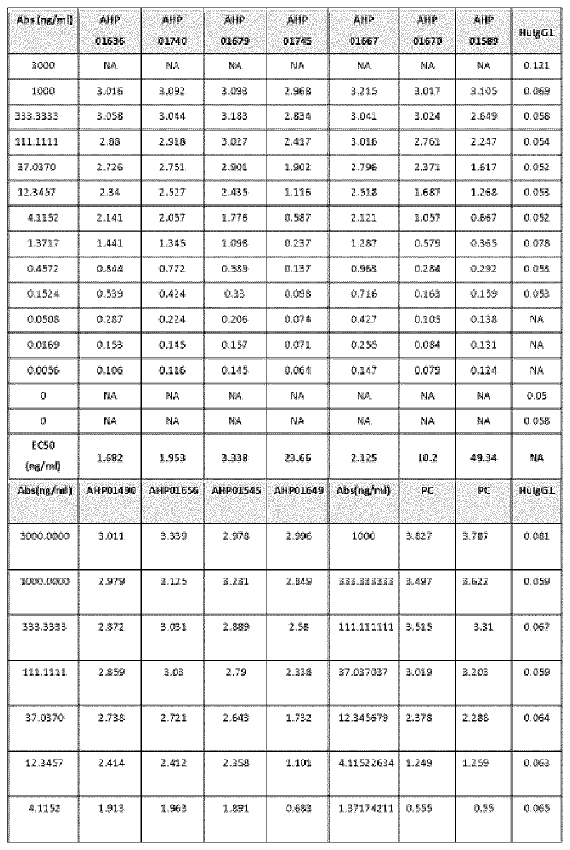

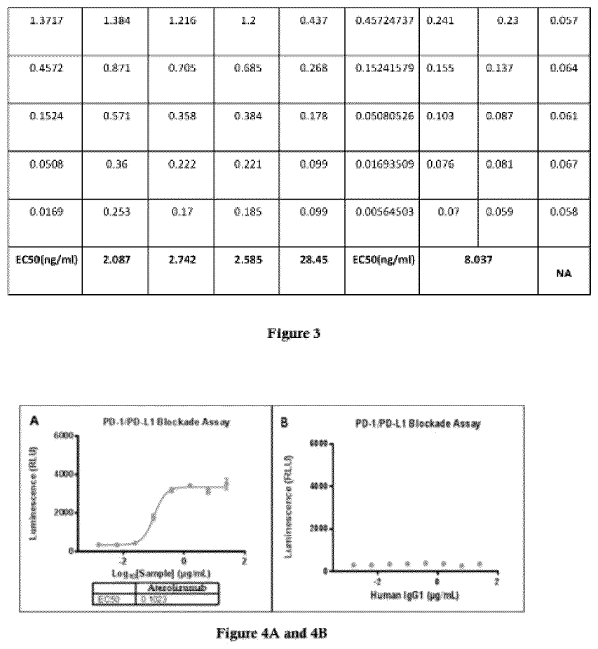

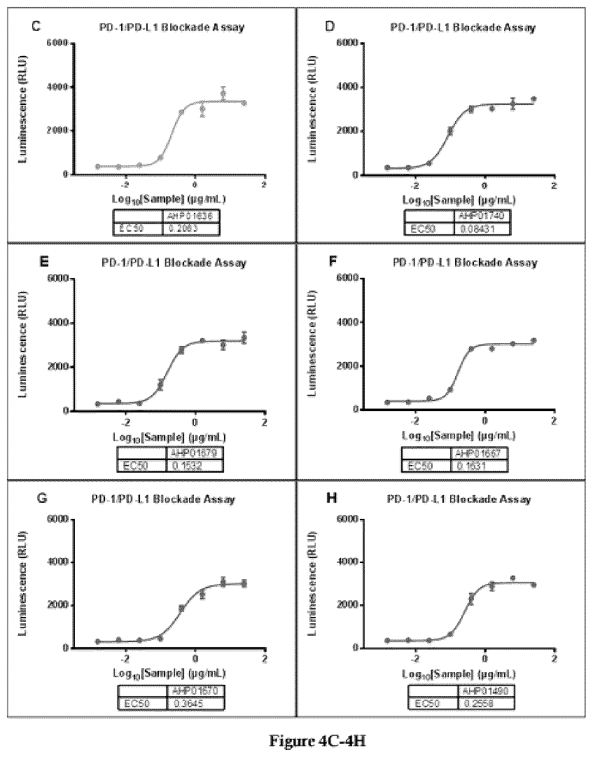

When positive control or test sample was added, they could block the interaction between PD-1 and PD-L1. Due to the blockade of either PD-1 or PD-L1, TCR activation, and NFAT-induced luciferase are able to release. The blockade activity of the anti-PD-L1 antibody was evaluated in terms of luciferase activity. FIG. 4 depicts the dose response curves plotted with the relative luminescence unit against the antibody concentration. Clearly the positive control, as depicted in FIG. 4A, showed blockade activity of PD-1/PD-L1 interaction, and all the selected nine IgGs, as depicted FIG. 4C-4H, showed blockade activity of PD-1/PD-L1 interaction, whereas, as depicted in FIG. 4B negative control human IgG did not show blockade activity.

Antibody candidate, AHP01679, was chosen as the antibody which showed best activity in vitro and was developed further for animal testing.

### 5. Selection of Final Antibody Candidate—AHP01679

AHP01679 candidate was finally chosen as best candidate antibody to show most efficient activity against PD-1/PD-L1 interaction.

AHP01679 candidate comprises:
a light chain of amino acid sequence of Seq. ID 6, and
a heavy chain of amino acid sequence of Seq. ID 12.

Seq. ID 6 comprises variable region consisting of three CDRs: LCDR1 of Seq. ID 1, LCDR2 of Seq. ID 3, and LCDR3 of Seq. ID 4.

Seq. ID 12 comprises variable region consisting of three CDRs: HCDR1 of Seq. ID 8; HCDR2 of Seq. ID 9; and HCDR3 of Seq. ID 10.

### Example 4

### Affinity Maturation of Antibody Candidate—AHP01679

AHP01679 candidate, also called the parental clone, was further used to generate more efficient antibodies by affinity maturation.

Nucleotide sequence encoding Fab regions of AHP01679 candidate sequence (light and heavy chain Fab sequences) were ligated to pAM vector to obtain the parental plasmid. Four single-site or double-site saturation mutagenesis libraries were designed. Seventy-two candidates (QJ001 candidates) with higher affinity than the parent candidate at the Fab level were obtained by means of three rounds of solid-phase panning and ELISA screening. Best of the ten high affinity QJ001 candidates were eventually selected to construct full-length antibody molecules with the sequence similarity to the 72 candidates, ELISA affinity analysis, and sequence modification sites taken into consideration. After expression and purification with CHO cell system, the constructed full-length antibody molecule underwent SDS and SEC purity tests, DSF thermal stability test, as well as affinity kinetics test and in vitro functional assay (CD3L-PD-L1-CHO and huPD-1-NF-AT-Jurkat cell-based assays) after CHO cell expression and purification.

Of the ten chosen QJ001 candidates QJ001-13 and QJ001-45 showed highest increase in affinity than the parental AHP01679 candidate.

Full length human antibodies of all the ten QJ001 candidates including QJ001-13 and QJ001-45 candidates were prepared for further analysis.

### Example 5

### Full Length Antibody Production and Analysis of Affinity Matured QJ001 Candidates

1. Full length antibody generation of affinity matured QJ001 candidates
The sequences were constructed into the pcDNA3.4 vector, followed by PCR, enzyme digestion, ligation, transformation, identification, sequencing, comparison, and extraction to obtain the plasmids.
Using ExpiCHO-s expression system, the above plasmids were expressed in CHO cells for 7 days as required, and the expression was measured by HPLC on day 6. Finally, quality control was conducted after purification and filling.

### 2. SDS PAGE Identification

Non-reduced and reduced samples of the full length QJ001 antibodies were analyzed by SDS PAGE electrophoresis. Purity of the reduced bands or purity of the reduced heavy chain plus the light chain was calculated using ImageJ via the peak area normalization method. The molecular weight of the reference IPI non-reduced band was about 150 kDa with purity >90%. The molecular weight was about 55 kDa for the reduced heavy chain and 25 kDa for the light chain, and the purity of the heavy chain plus the light chain was >90%.

### Table 6 Shows the Expression Levels and Purity of QJ001 Antibodies

| | | | Expression | | Non- | |
|---|---|---|---|---|---|---|
| | | | on CHO | | Reduced | |
| Sample | Sample | Protein | cells | MW | purity | Reduced |
| No. | name | No. | (μg/mL) | (kDa) | (%). | purity(%). |
| 1 | QJ001-13 | P02892 | 77.30 | 143.68 | <90.0 | >95.0 |
| 2 | QJ001-15 | P02893 | 79.50 | 143.78 | <90.0 | >95.0 |
| 3 | QJ001-29 | P02894 | 53.20 | 143.74 | <90.0 | >95.0 |
| 4 | QJ001-32 | P02895 | 42.20 | 143.56 | <90.0 | >95.0 |
| 5 | QJ001-37 | P02896 | 36.40 | 143.48 | <90.0 | >95.0 |
| 6 | QJ001-45 | P02897 | 60.30 | 143.74 | <90.0 | >95.0 |
| 7 | QJ001-46 | P02898 | 38.40 | 143.5 | <90.0 | >95.0 |
| 8 | QJ001-47 | P02899 | 55.30 | 143.74 | <90.0 | >95.0 |
| 9 | QJ001-59 | P02900 | 67.00 | 144.02 | <90.0 | >95.0 |
| 10 | QJ001-62 | P03559 | 237.00 | 144.02 | <90.0 | >90.0 |
| 11 | Parental | P03560 | 233.00 | 143.72 | <90.0 | >90.0 |
| | Ab/ | | | | | |
| | AHP01679 | | | | | |

### Example 6

### Affinity Kinetics Analysis of QJ001 Candidates

Affinity kinetics of affinity matured antibody candidates QJ001 were compared with parental candidate antibody AHP01679, and positive control antibody, Atezolizumab using GATOR instrument and related software to conduct Kinetics experiment.

| | | KD (Parental | | | | | |
|---|---|---|---|---|---|---|---|
| | | Ab)/KD | HuPD-L1-his | | | | |
| S. | | (Candidate | | | | | Rmax |
| no. | Sample name | Ab) | KD (M) | ka (1/Ms) | kd (1/s) | R² | (nm) |
| 1 | Atezolizumab | 16.96 | 2.07E−11 | 4.11E+06 | 8.51E−05 | 0.983 | 0.486 |
| 2 | Parental Ab | 1.00 | 3.51E−10 | 3.77E+06 | 1.32E−03 | 0.98 | 0.509 |
| 3 | QJ001-13 | 27.64 | 1.27E−11 | 4.87E+06 | 6.19E−05 | 0.98 | 0.501 |
| 4 | QJ001-15 | 7.36 | 4.77E−11 | 6.14E+06 | 2.93E−04 | 0.99 | 0.561 |
| 5 | QJ001-29 | 3.25 | 1.08E−10 | 4.28E+06 | 4.64E−04 | 0.986 | 0.447 |
| 6 | QJ001-32 | 5.66 | 6.20E−11 | 4.33E+06 | 2.69E−04 | 0.983 | 0.485 |
| 7 | QJ001-37 | 5.46 | 6.43E−11 | 3.53E+06 | 2.27E−04 | 0.989 | 0.392 |
| 8 | QJ001-45 | 10.38 | 3.38E−11 | 4.29E+06 | 1.45E−04 | 0.984 | 0.557 |
| 9 | QJ001-46 | 3.91 | 8.97E−11 | 4.48E+06 | 4.02E−04 | 0.987 | 0.475 |
| 10 | QJ001-47 | 6.71 | 5.23E−11 | 5.08E+06 | 2.66E−04 | 0.991 | 0.517 |
| 11 | QJ001-59 | 6.78 | 5.18E−11 | 6.91E+06 | 3.58E−04 | 0.990 | 0.510 |

Table 7 Provides the KD Affinity Values of Selected anti-PD-L1 Antibodies Against Parental Antibody AHP01679

Table 7 above provides the KD affinity values. KD (Parental Ab)/KD (candidate antibody) value determines the fold times the affinity of candidate antibody towards target antigen is stronger than the parental antibody AHP01679.

It is clearly visible that QJ001-13 and QJ001-45 candidate antibodies showed the highest affinity. QJ001-13 showed 27 times higher affinity compared to parental antibody AHP01679, and QJ001-45 showed 10 times higher affinity.

The study results showed that the correlation coefficients R2 of all antibodies was greater than 0.95 in the Global fitting mode, which met the requirements and the results were reliable. Hence, for animal model studies QJ001-13 and QJ001-45 candidates were chosen along with the parental antibody AHP01679.

### Example 7

### Therapeutic Activity Studies Using Animal Model

Parental antibody AHP01679, and affinity matured QJ001-13 and QJ001-45 candidates were tested for their efficiency in reducing tumor growth in mouse model.

The evaluation of drug efficacy at the preclinical stage requires animal models allowing extrapolation to humans. Humanized mice, which stably and autonomously maintain human tissues/proteins, are useful for filling the species gap between humans and animals.

To test the in vivo efficacy of anti-PD-L1 monoclonal antibodies, murine colon cancer model based on hPD-L1-MC38 colon cell line in humanized B-hPD-1/hPD-L1 mice were employed. In brief, B-hPD-1/hPD-L1 mice were subcutaneously injected with MC38-hPD-L1 tumor cells (5×10⁵) suspended in 0.1 mL PBS in the right front flank for tumor development. Tumor-bearing animals were randomly enrolled into 4 study groups when the mean tumor size reached 109 mm³. Each group consisted of 6 mice. The treatment groups were as follows;
a. Isotype control (human IgG1) (10 mg/kg),
b. AHP01679 (10 mg/kg),
c. QJ001-13 (10 mg/kg),
d. QJ001-45 (10 mg/kg),
e. Atezolizumab (10 mg/kg).

Dosing frequency was BIW (twice per week) following intraperitoneal route. In this experiment, all test articles under the tested doses presented no negative effect on animal body weight or obvious clinical sign.

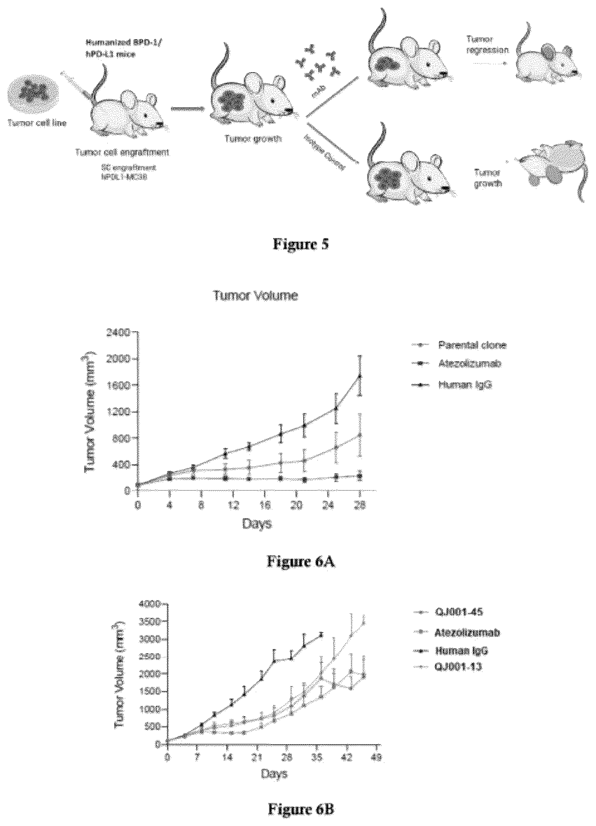

FIG. 5 depicts a schematic of the experimental strategy to determine the therapeutic activity of said antibodies. Briefly, tumor cells were injected in humanized mouse model for PD-L1 for development of tumor growth. After the development of detectable tumor, monoclonal human antibodies were injected in the mice. Further positive control antibodies (Atezolizumab) and negative control antibodies (HIgGs) were used for assessment. The negative control mice are expected to showed uncontrolled tumor growth and subsequent death after tumor progression. Whereas, a therapeutic antibody will reduce the size of the tumor and result in tumor regression.

As depicted in FIG. 6A, the parental clone AHP01679 reduced tumor growth significantly, as did the positive control Atezolizumab, compared to uncontrolled tumor growth in negative control mice. Further, FIG. 6B compares effect of QJ001-45, and QJ001-13 affinity matured candidates. Initially QJ001-45, and QJ001-13 showed almost similar efficiency, however, after 35 days tumor showed progression in QJ001-13 injected mice. Between 42-45 days, tumor growth in QJ001-13 injected mice was equivalent to that of negative control human IgG injected mice. Whereas, tumor growth in QJ001-45 injected mice was consistently and significantly reduced and was comparable to the positive control Atezolizumab.

To conclude, even though QJ001-13 showed better affinity and PD-1/PDL1 blockade activity in vitro compared to QJ001-45 candidate, QJ001-45 was more efficient in reducing tumor growth in in vivo studies. Hence, QJ001-45 affinity matured candidate was finalized as the most efficient affinity matured antibody derived from parental candidate AHP01679.

### Example 8

### QJ001-45 Affinity Matured Antibody

QJ001-45 affinity matured candidate comprises:
a light chain of amino acid sequence of Seq. ID 7, and
a heavy chain of amino acid sequence of Seq. ID 13.

Seq. ID 7 comprises variable region consisting of three CDRs: LCDR1 of Seq. ID 2, LCDR2 of Seq. ID 3, and LCDR3 of Seq. ID 5.

Seq. ID 13 comprises variable region consisting of three CDRs: HCDR1 of Seq. ID 8; HCDR2 of Seq. ID 9; and HCDR3 of Seq. ID 11.

### Example 9

### PD1-PD-L1 Blockade Assay

Further, functional activity of selected antibodies, parental AHP01679 and affinity matured

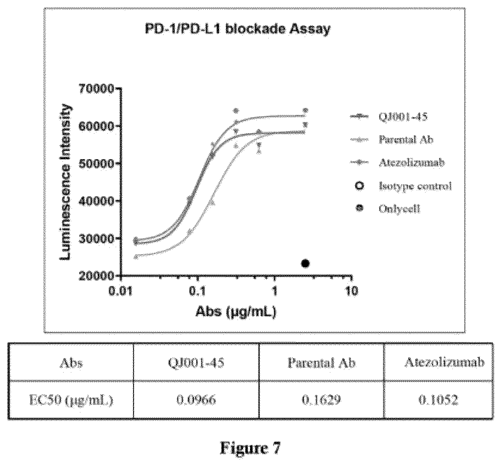

QJ001-45, was tested against the positive control Atezolizumab by PD1/PD-L1 blockade bioassay. As shown in FIG. 7, the efficiency of parental AHP01679 antibody was comparable to the positive control, and affinity matured QJ001-45 showed improved efficiency over the parental antibody.

While certain exemplary embodiments have been described and shown in the accompanying drawings, it is to be understood that such embodiments are merely illustrative of, and not restrictive on, the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein

We claim:

1. A human antibody or antigen binding fragment specific against PD-L1 comprising
a) a light chain variable region comprising of LCDR1, LCDR2, and LCDR3 sequences, and
b) a heavy chain variable region comprising of HCDR1, HCDR2, and HCDR3 sequences,
Wherein,
LCDR1 is amino acid sequence selected from the group consisting of SEQ ID NO: 1 or SEQ ID NO: 2;
LCDR2 is amino acid sequence of SEQ ID NO: 3;
LCDR3 is amino acid sequence selected from the group consisting of SEQ ID NO: 4 or SEQ ID NO: 5;
HCDR1 is amino acid sequence of SEQ ID NO: 8;
HCDR2 is amino acid sequence of SEQ ID NO: 9; and
HCDR3 is amino acid sequence selected from the group consisting of SEQ ID NO: 10 or SEQ ID NO: 11.

2. The human antibody as claimed in claim 1, wherein, the light chain variable region comprises of LCDR1 of SEQ ID NO: 1, LCDR2 is of SEQ ID NO: 3, and LCDR3 is of SEQ ID NO: 4.

3. The human antibody as claimed in claim 1, wherein, the light chain variable region comprises of LCDR1 of SEQ ID NO: 2, LCDR2 is of SEQ ID NO: 3, and LCDR3 is of SEQ ID NO: 5.

4. The human antibody as claimed in claim 1, wherein, the heavy chain variable region comprises of HCDR1 of SEQ ID NO: 8, HCDR2 is of SEQ ID NO: 9, and HCDR3 is of SEQ ID NO: 10.

5. The human antibody as claimed in claim 1, wherein, the heavy chain variable region comprises of HCDR1 of SEQ ID NO: 8, HCDR2 is of SEQ ID NO: 9, and HCDR3 is of SEQ ID NO: 11.

6. A human antibody or antigen binding fragment specific against PD-L1, comprising
a) a light chain sequence, and
b) a heavy chain sequence;
wherein
the light chain sequence comprises of amino acid sequence selected from the group consisting of SEQ ID NO: 6 and SEQ ID NO: 7; and
the heavy chain sequence comprises of amino acid sequence selected from the group consisting of SEQ ID NO: 12 and SEQ ID NO: 13.

7. The human antibody as claimed in claim 6, wherein the light chain of SEQ ID NO: 6 consists of LCDR1 of SEQ ID NO: 1, LCDR2 of SEQ ID NO: 3, and LCDR3 of SEQ ID NO: 4.

8. The human antibody as claimed in claim 6, wherein the light chain of SEQ ID NO: 7 consists of LCDR 1 of SEQ ID NO: 2, LCDR2 of SEQ ID NO: 3, and LCDR3 of SEQ ID NO: 5.

9. The human antibody as claimed in claim 6, wherein the heavy chain of SEQ ID NO: 12 consists of HCDR1 of SEQ ID NO: 8; HCDR2 of SEQ ID NO: 9; and HCDR3 of SEQ ID NO: 10.

10. The human antibody as claimed in claim 6, wherein the heavy chain of SEQ ID NO: 13 consists of HCDR1 of SEQ ID NO: 8; HCDR2 of SEQ ID NO: 9; and HCDR3 of SEQ ID NO: 11.

11. The human antibody as claimed in claim 6, wherein, the light chain is amino acid sequence of SEQ ID NO: 6, and heavy chain is amino acid sequence of SEQ ID NO: 12 having a dissociation constant of 4.09*10⁻⁹ M, and an EC₅₀ value 0.1629 μg/ml.

12. The human antibody as claimed in claim 6, wherein, the light chain is amino acid sequence of SEQ ID NO: 7 and heavy chain is amino acid sequence of SEQ ID NO: 13 having a dissociation constant of 0.338*10⁻¹² M and an EC₅₀ value 0.0966 μg/ml.

13. The human antibody or antigen binding fragment specific against PD-L1 as claimed in claim 6 for use in the treatment of cancer or infectious diseases.

14. A pharmaceutical composition comprising the human antibody or antigen binding fragment specific against PD-L1 as claimed in claim 6, and at least a pharmaceutically acceptable carrier or excipient.


SEQUENCE LISTING (17 sequences)

SEQ ID NO: 1 - protein, 14 aa, synthetic construct
  REGION 1..14: note = LCDR1
TGSSSNIGAGYDVH

SEQ ID NO: 2 - protein, 14 aa, synthetic construct
  REGION 1..14: note = LCDR1
FGSSSNIGAGYDVH

SEQ ID NO: 3 - protein, 8 aa, synthetic construct
  REGION 1..8: note = LCDR2
YGNNNRPS

SEQ ID NO: 4 - protein, 11 aa, synthetic construct
  REGION 1..11: note = LCDR3
QSYDSSLSGSV

SEQ ID NO: 5 - protein, 11 aa, synthetic construct
  REGION 1..11: note = LCDR3
GSYDSSLSGSV

SEQ ID NO: 6 - protein, 236 aa, synthetic construct
  REGION 1..236: note = AHP01679-LC
MGWSCIILFLVATATGVHSQAVLTQPSSVSGAPGQRVTISCTGSSSNIGAGYDVHWYQQLPGTAPKLLIYGNNNRPSGVPDRFSGSKSGTSASLAITGLQAEDEADYYCQSYDSSLSGSVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 7 - protein, 217 aa, synthetic construct
  REGION 1..217: note = QJ001-45-LC
QAVLTQPSSVSGAPGQRVTISCFGSSSNIGAGYDVHWYQQLPGTAPKLLIYGNNNRPSGVPDRFSGSKSGTSASLAITGLQAEDEADYYCGSYDSSLSGSVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 8 - protein, 10 aa, synthetic construct
  REGION 1..10: note = HCDR1
GGTFSSYAIS

SEQ ID NO: 9 - protein, 13 aa, synthetic construct
  REGION 1..13: note = HCDR2
WMGRIIPILGIAN

SEQ ID NO: 10 - protein, 13 aa, synthetic construct
  REGION 1..13: note = HCDR3
TTFIQQWLYAFDI

SEQ ID NO: 11 - protein, 13 aa, synthetic construct
  REGION 1..13: note = HCDR3
TTFFQQWLYAFDI

SEQ ID NO: 12 - protein, 469 aa, synthetic construct
  REGION 1..469: note = AHP01679 HC
MGWSCIILFLVATATGVHSEVQLVQSGAEVKKPGSSVKVSCKASGGTFSSYAISWVRQAPGQGLEWMGRIIPILGIANYAQKFQGRVTITADKSTSTAYMELSSLRSEDTAVYYCTTFIQQWLYAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 450 aa, synthetic construct
  REGION 1..450: note = QJ001-45 HC
EVQLVQSGAEVKKPGSSVKVSCKASGGTFSSYAISWVRQAPGQGLEWMGRIIPILGIANYAQKFQGRVTITADKSTSTAYMELSSLRSEDTAVYYCTTFFQQWLYAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 654 bp, synthetic construct
  misc_feature 1..654: note = AHP01679-LC
  source 1..654: mol_type = other DNA
caggccgtgctgacccagcctagctccgtgtctggagcaccaggacagagggtgaccatcagctgcacaggctctagctccaatatcggcgccggctacgacgtgcactggtatcagcagctgcccggcacagcccctaagctgctgatctacggcaacaataaccggccatccggcgtgcccgatagattctccggctctaagagcggcacctccgcctctctggccatcacaggactgcaggcagaggacgaggcagattactattgtcagtcttatgactctagcctgagcggatccgtgtttggaggaggaaccaagctgacagtgctggggcagcccaaggccgctcctagcgtgacactgtttcccccttcctccgaggagctgcaggccaacaaggccaccctggtgtgcctgatctccgacttctatcctggcgccgtgacagtggcctggaaggctgattctagcccagtgaaggctggcgtggagaccacaaccccctccaagcagtctaacaataagtatgccgcttcctcttacctgagcctgacaccagagcagtggaagtcccaccggtcttacagctgccaggtcactcacgaaggctctaccgtggaaaagacagtcgcacccaccgaatgctcatga

SEQ ID NO: 15 - DNA, 1353 bp, synthetic construct
  misc_feature 1..1353: note = AHP01679-HC
  source 1..1353: mol_type = other DNA
gaggtgcagctggtgcagagcggagcagaggtgaagaagccaggaagctccgtgaaggtgtcctgcaaggcctctggcggcaccttctctagctacgccatctcctgggtgcggcaggcaccaggacagggactggagtggatgggcagaatcatccctatcctgggcatcgccaactacgcccagaagtttcagggcagggtgaccatcacagccgacaagtctaccagcacagcctatatggagctgtcctctctgcggtctgaggatacagccgtgtactattgtaccacattcatccagcagtggctgtatgcctttgacatctggggccagggcaccatggtgacagtgagctccgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtga

SEQ ID NO: 16 - DNA, 651 bp, synthetic construct
  misc_feature 1..651: note = QJ001-45-LC
  source 1..651: mol_type = other DNA
caggctgttctgacccagccttcctctgtgtctggtgctcctggccagagagtgaccatctcttgttttggctcctcctccaatatcggcgctggctacgacgtgcactggtatcagcagctgcctggcacagctcccaaactgctgatctacggcaacaacaaccggccttctggcgtgcccgatagattctccggctctaagtctggcacctctgccagcctggctatcactggactgcaggctgaggacgaggccgactactactgcgggtcctacgactcttccctgtccggctctgtttttggcggaggcaccaagctgacagtgctgggacagcctaaggctgccccttccgtgacactgttccctccatcctctgaggaactgcaggccaacaaggctaccctcgtgtgcctgatctccgacttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccaccggtcctacagctgccaagtgacccatgagggctccaccgtggaaaagaccgtggctcctaccgagtgctct

SEQ ID NO: 17 - DNA, 2601 bp, synthetic construct
  misc_feature 1..2601: note = QJ001-45-HC
  source 1..2601: mol_type = other DNA
gaagttcagctggttcagtctggcgccgaagtgaagaaacctggctcctccgtgaaggtgtcctgcaaggcttctggcggcaccttctcctcttacgccatctcctgggtccgacaggctcctggacaaggcttggagtggatgggcagaatcatccccatcctgggaatcgccaactacgcccagaaattccagggcagagtgaccatcaccgccgacaagtctacctccaccgcctacatggaactgtccagcctgagatctgaggacaccgccgtgtactactgcaccaccttctttcagcagtggctgtacgccttcgacatctggggccagggcacaatggtcaccgtgtcctctgcttccaccaagggcccctccgtgttccccctggctccctcttccaagagcaccagcggcggcaccgctgctctgggatgtctggtgaaggactacttccctgagcctgtgaccgtgtcctggaattccggcgccctgacctccggcgtgcacacattccctgctgtgctgcagtcctccggcctgtatagcctgtcctccgtggtgacagtgcctagctccagcctgggcacccagacctatatctgcaacgtgaaccacaagcctagcaataccaaggtggacaagaaggtggagcctaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaaggaagttcagctggttcagtctggcgccgaagtgaagaaacctggctcctccgtgaaggtgtcctgcaaggcttctggcggcaccttctcctcttacgccatctcctgggtccgacaggctcctggacaaggcttggagtggatgggcagaatcatccccatcctgggaatcgccaactacgcccagaaattccagggcagagtgaccatcaccgccgacaagtctacctccaccgcctacatggaactgtccagcctgagatctgaggacaccgccgtgtactactgcaccaccttctttcagcagtggctgtacgccttcgacatctggggccagggcacaatggtcaccgtgtcctctgcttccaccaagggcccctccgtgttccccctggctccctcttccaagagcaccagcggcggcaccgctgctctgggatgtctggtgaaggactacttccctgagcctgtgaccgtgtcctggaattccggcgccctgacctccggcgtgcacacattccctgctgtgctgcagtcctccggcctgtatagcctgtcctccgtggtgacagtgcctagctccagcctgggcacccagacctatatctgcaacgtgaaccacaagcctagcaataccaaggtggacaagaaggtggagcctaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggcaaa